(19) United States
(12) Patent Application Publication
(10) Pub. No.: US 2022/0178856 A1
(43) Pub. Date: Jun. 9, 2022

(54) CATALYST FOR A GAS SENSOR AND A CONTACT COMBUSTION TYPE GAS SENSOR HAVING THE SAME

(71) Applicants: HYUNDAI MOTOR COMPANY, Seoul (KR); KIA CORPORATION, Seoul (KR)

(72) Inventor: Byung Jin Jang, Suwon-si (KR)

(73) Assignees: HYUNDAI MOTOR COMPANY, Seoul (KR); KIA CORPORATION, Seoul (KR)

(21) Appl. No.: 17/515,042

(22) Filed: Oct. 29, 2021

(30) Foreign Application Priority Data
Dec. 8, 2020 (KR) 10-2020-0170857

(51) Int. Cl. G01N 25/22; B01J 23/42; B01J 21/06; B01J 35/10; B01J 35/00; G01N 33/00

(52) U.S. Cl. CPC G01N 25/22; B01J 23/42; B01J 21/06; B01J 35/1009; G01N 33/005; B01J 35/1019; B01J 35/0006; B01J 35/1061; B01J 35/1057; B01J 35/1014

(57) ABSTRACT

A catalyst for a gas sensor includes a support and a core-shell type complex contained in the support. The complex includes a metal-containing core and a porous nanostructured shell. A contact combustion type gas sensor includes the catalyst.

### CROSS-REFERENCE TO RELATED APPLICATION

This application claims the benefit of priority to Korean Patent Application No. 10-2020-0170857, filed in the Korean Intellectual Property Office on Dec. 8, 2020, the entire contents of which are incorporated herein by reference.

### TECHNICAL FIELD

The present disclosure relates to a catalyst for a gas sensor that lasts a long time and has excellent durability, and a contact combustion type gas sensor including the same.

### BACKGROUND

Gas sensors are classified into a physicochemical method, an optical method, an electrical method, or the like based on a detection manner. A sensor based on a physicochemical principle includes a semiconductor type, an electrochemical type, a contact combustion type, and the like. In particular, a hydrogen sensor that detects hydrogen, which is a combustible gas, is an essential sensor for devices that use hydrogen energy. The hydrogen sensor detects a hydrogen gas leak or continuously monitors a concentration of hydrogen gas in the air in a closed system, thereby preventing explosion by hydrogen gas.

In detail, the contact combustion type hydrogen sensor detects hydrogen gas by converting reaction heat (heat of combustion) of oxygen and hydrogen, which are combustible gases, into an electric signal. Accordingly, the contact combustion type hydrogen sensor has excellent stability and durability because it is relatively less affected by water vapor, temperature, humidity, and other gases. The contact combustion type hydrogen sensor generally includes a support made of porous ceramics capable of uniformly containing a large amount of catalyst and a metal heating wire in the support. The support includes a catalyst. The contact combustion type hydrogen sensor as described above heats the support by applying power to the metal heating wire and a combustion reaction occurs when hydrogen gas comes into contact with the heated support. The temperature of the support and the metal heating wire in the support is increased by the combustion reaction and a resistance value of the metal heating wire is changed, thereby detecting the hydrogen gas.

For example, Korean Patent Application Publication No. 2010-0026810 (Patent Document 1) discloses a chip manufactured using micro electro-mechanical system (MEMS) technology and a contact combustion type hydrogen sensor including a catalyst in which platinum (Pt) is highly dispersed on an alumina carrier. However, the conventional contact combustion type hydrogen sensor disclosed in Patent Document 1 uses combustion of the catalyst Thus, the combustion reaction is weakened due to deterioration of the catalyst during long-term use, thereby gradually decreasing sensitivity. In addition, in Patent Document 1, as a carrier in which metal particles are dispersed deteriorates, agglomeration due to movement of metal particles in the carrier and growth of metal particles thereby may occur, or desorption of metal particles in the carrier may occur. In particular, the movement and growth of metal particles may be accelerated by the external environment, i.e., temperature and moisture. The movement and growth of the metal particles reduce the area of the metal particles capable of reacting with hydrogen gas, thereby reducing performance of the sensor.

In addition, as moisture (H₂O) present in the external and surrounding environment acts as a forward reaction and a competitive reaction of the contact combustion type hydrogen sensor, activity of the sensor is lowered. Also, the catalyst causes transformation through reaction with a sensing target material to be detected. Life expectancy of the sensor is thereby shortened. The shortened life expectancy causes additional costs such as sensor replacement or certification/calibration.

Therefore, there is a need for research and development of a catalyst for the gas sensor with increased sensor life and excellent durability. These are achieved by preventing deformation by moisture existing in the external and surrounding environment and by preventing movement and growth of the metal particles due to deterioration. There is also a need for research and development of a contact combustion type gas sensor including the same.

### SUMMARY

The present disclosure has been made to solve the above-mentioned problems occurring in the prior art while advantages achieved by the prior art are maintained intact.

An aspect of the present disclosure provides a catalyst for a gas sensor with a long sensor life and excellent durability. These aspects are achieved by preventing deformation by moisture existing in the external and surrounding environment and by preventing movement and growth of metal particles due to deterioration. Another aspect of the present disclosure provides a contact combustion type gas sensor including the same.

The technical problems to be solved by the present inventive concept are not limited to the aforementioned problems, and any other technical problems not mentioned herein will be clearly understood from the following description by those having ordinary skill in the art to which the present disclosure pertains.

According to an aspect of the present disclosure, a catalyst for a gas sensor includes a support and a core-shell type complex contained in the support.

The complex includes a metal-containing core and a porous nanostructured shell.

In addition, according to an aspect of the present disclosure, a contact combustion type hydrogen sensor includes the catalyst for the gas sensor.

### BRIEF DESCRIPTION OF THE DRAWINGS

The above and other objects, features, and advantages of the present disclosure should be more apparent from the following detailed description taken in conjunction with the accompanying drawings:

FIG. 1 is a schematic diagram of a catalyst for a gas sensor according to an embodiment of the present disclosure; and

FIGS. 2 and 3 are results of measuring a current difference for hydrogen gas in Experimental Examples 1 and 2 targeting a catalyst for a gas sensor according to an embodiment of the present disclosure.

### DETAILED DESCRIPTION

Hereinafter, the present disclosure is described in detail.

Catalyst for Gas Sensor

A catalyst for a gas sensor according to the present disclosure includes a support and a core-shell type complex contained in the support.

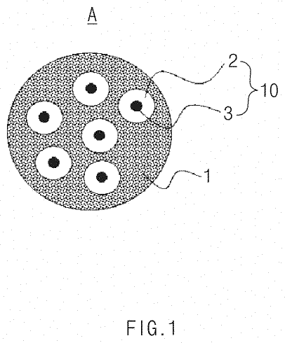

Referring to FIG. 1, a catalyst “A” for a gas sensor according to the present disclosure may include a support “1” and a core-shell type complex 10 contained in the support “1”.

Support

The support contains the complex. Thus, the complex is dispersed and mechanically fixed in the support to increase catalytic activity, thereby serving to increase thermal conductivity.

The support may be a ceramic containing a metal oxide. For example, the ceramic may include at least one selected from a group consisting of aluminum (Al), silicon (Si), boron (B), beryllium (Be), tin (Sn), zinc (Zn), tungsten (W), copper (Cu), and magnesium (Mg). In detail, the ceramic may be formed of at least one selected from a group consisting of aluminum oxide (Al₂O₃), aluminum nitride (AlN), silicon carbide (SiC), silicon nitride (Si₃N₄), boron nitride (BN), beryllium oxide (BeO), tin(IV) oxide (SnO₂), zinc peroxide (ZnO₂), tungsten trioxide (WO₃), copper oxide (CuO), and magnesium oxide (MgO).

The support may include a carbon-based compound. For example, the support may further include at least one selected from a group consisting of carbon black, carbon fiber, graphite, carbon nanotubes, and graphene.

Core-shell Type Complex

The core-shell type complex is contained in the support and includes a metal. Thus, the complex serves to detect a target gas by accelerating a reaction between the target gas and oxygen.

The complex includes a metal-containing core and a porous nanostructured shell. Referring to FIG. 1, the complex 10 may include a metal-containing core “3” and a porous nanostructured shell “2”. When the complex is in a form of a core-shell agglomeration, in which metal particles move in the support and aggregate with one another, one of the representative deterioration phenomena of a catalyst, is prevented and contact with external moisture is prevented. This results in having less change in the sensitivity of the sensor. In addition, the core-shell type complex may be regenerated as moisture in the complex is evaporated for improvement of catalytic activity by heat and may be reused.

Here, the porous nanostructured shell serves to uniformly disperse the metal-containing core in the support and serves to prevent the sensor sensitivity from being lowered. This is due to deterioration of metal-containing core, aggregation due to particle movement through reaction with water, particle growth thereby, or desorption of the metal particle.

The shell may include a plurality of pores each having an average diameter of less than 10 nm, 1 to 10 nm, or 2 to 5 nm. When the average diameter of the pores in the shell is less than the above range, a sensing target gas does not move smoothly and thus the sensitivity of the sensor may be insufficient due to transmittance. When the average diameter exceeds the above range, permeability of large gas particles including moisture particles increases and thus the sensitivity of the sensor may be deteriorated due to the moisture.

In addition, the shell may include at least one selected from a group consisting of silica, zeolite, and starch.

The shell may have an average thickness of 10 to 100 nm, or 10 to 50 nm. When the average thickness of the shell is less than the above range, a filtering effect may be degraded because there is no discrimination in permeability of gas. When the average thickness of the shell exceeds the above range, the filtering effect may be degraded because the gas cannot be smoothly moved.

The metal-containing core serves to accelerate a reaction between the target gas and oxygen.

In addition, the metal may include at least one selected from a group consisting of platinum (Pt), palladium (Pd), gold (Au), nickel (Ni), ruthenium (Ru), copper (Cu), and rhodium (Rh).

The core may have an average diameter of 1 to 12 nm, or 2 to 6 nm. When the average diameter of the core is less than the above range, the lifetime of the sensor may be reduced due to deterioration of the catalyst. When the average diameter of the core exceeds the above range, reactivity degradation may occur due to catalyst agglomeration or reduced surface area.

The complex may be synthesized by a templating method. For example, the complex may be synthesized by a templating method including coating a template such as an emulsifier, colloid, block copolymer, emulsion, micro-molding, or the like with the porous nanostructured shell as described above, preparing a hollow porous nanostructured shell by removing the template, and containing the metal on the core that is the inside of the shell of the hollow porous nanostructure.

In addition, the complex may include a lipophilic functional group on a surface thereof. When the complex includes the lipophilic functional group on the surface thereof, there is a waterproof effect that prevents penetration of moisture particles.

More specifically, the lipophilic functional group may be derived from at least one selected from a group consisting of aluminic ester and aluminum hydroxide. For example, the complex may include the lipophilic functional group on the surface thereof by surface modification treatment with at least one selected from the group consisting of aluminic ester and aluminum hydroxide.

The complex may include a hydrophobic functional group on the surface thereof. When the complex includes the hydrophobic functional group on the surface thereof, moisture particles are prevented from penetrating into the core, and thus durability of the catalyst is improved.

The catalyst for the gas sensor may have a Brunauer, Emmett, and Teller (BET) specific surface area of 50 to 300 m²/g, or 100 to 250 m²/g. When the BET specific surface area of the catalyst is less than the above range, the reaction area of the catalyst is small, and thus reactivity with the target gas may be lowered. When the catalyst is more than the above range, the catalyst reactivity may be too high and may be susceptible to deterioration, thereby reducing catalyst lifetime.

In addition, the catalyst for the gas sensor may include a lipophilic functional group derived from at least one selected from a group consisting of aluminic ester and aluminum hydroxide on the surface thereof. For example, the catalyst may include the lipophilic functional group on the surface thereof by surface modification treatment with at least one selected from the group consisting of aluminic ester and aluminum hydroxide.

The catalyst for the gas sensor may include a hydrophilic functional group on the surface thereof. For example, the catalyst for the gas sensor may include a hydrophilic functional group and a lipophilic functional group on the surface thereof.

In addition, the catalyst for the gas sensor may have an average diameter of 20 to 500 nm. Here, the catalyst for the gas sensor may be formed by mixing a plurality of catalysts having different average diameters. For example, the catalyst for the gas sensor may be formed by mixing catalysts having an average diameter of 20 nm, 50 nm, 200 nm, and 500 nm, respectively.

As described above, the catalyst for the gas sensor according to the present disclosure prevents deformation due to moisture existing in the external and surrounding environment. The catalyst also prevents movement and growth of metal particles due to deterioration, and thus the sensor has a long lifetime and excellent durability.

Contact Combustion Type Gas Sensor

The contact combustion type gas sensor according to the present disclosure includes the catalyst for the gas sensor as described above.

The contact combustion type gas sensor may be manufactured in a micro electro-mechanical system (MEMS). For example, the sensor may have a structure in which a device electrode, an insulating layer, and a catalyst layer including a catalyst for a gas sensor are stacked.

In addition, the contact combustion type gas sensor may detect at least one gas selected from a group consisting of hydrogen gas, ethanol gas, formaldehyde gas, and hydrocarbon (CₓHₓ) gas. Here, the hydrocarbon gas may be, for example, methane, isobutane, or the like.

The contact combustion type gas sensor may be applied as a hydrogen sensor for a hydrogen electric vehicle, an alcohol sensor for a vehicle, an indoor air quality sensor for a vehicle, or the like.

The contact combustion type gas sensor according to the present disclosure as described above is very suitable for application to a hydrogen sensor that detects a concentration of hydrogen gas in a hydrogen electric vehicle and checks whether there is leakage, an alcohol sensor that measures whether a driver is drunk, and an indoor air quality sensor that measures a concentration of a volatile organic compound (VOC) in the vehicle.

Hereinafter, the present disclosure is described in more detail through examples. However, these examples are only intended to aid understanding of the present disclosure and the scope of the present disclosure is not limited to these examples in any sense.

### EXAMPLES

### Example 1

35.5 ml of deionized water and 1.0 ml of an aqueous sodium hydroxide solution having a concentration of 0.05M were added to an aqueous solution of colloidal Pt nanoparticles dispersed in 5 ml of deionized water (concentration 4.5×10⁻⁵ mol). A pH was adjusted to 10 to 11. Then, methanol containing tetraethyl orthosilicate (TEOS) of 10% by volume was added and calcined at 350° C. for 2 hours to prepare a core Pt-shell silicate complex. Thereafter, the complex and alumina (γ-Al₂O₃) in powder form were mixed to contain the complex in alumina to prepare a catalyst for a gas sensor.

### Example 2

35.5 ml of deionized water and 1.0 ml of an aqueous sodium hydroxide solution having a concentration of 0.05M were added to an aqueous solution of colloidal Pt nanoparticles dispersed in 5 ml of deionized water (concentration 4.5×10⁻⁵ mol). A pH was adjusted to 10 to 11. Then, methanol containing tetraethyl orthosilicate (TEOS) of 10% by volume was added and was stirred for 2 hours to become homogeneous, thereby preparing a stirring solution containing the complex consisting of the core Pt-shell silicate.

After the stirring solution was added drop by drop to γ-alumina powder (99.9%) and dried for a day in air at 150° C. The dried powder was pulverized. The pulverized powder was heated in an oven at 300° C. for 2 hours and then cooled to prepare a catalyst for a gas sensor.

### Example 3

Ammonia water was mixed at 30% by weight with ethanol and deionized water. TEOS and octadecyl trimethoxysilane (OTMS) were added in a 3:1 ratio and stirred for 6 hours to prepare a spherical silica template. The prepared spherical silica template was separated from the solution by centrifugation and calcined at 500° C.

Then, the spherical silica template was put in an ethanol solution of hexachloroplatinic acid hexahydrate (H₂PtCl₆·6H₂O) and probed through ultrasonic treatment for 1 hour to prepare a core Pt-shell silicate complex.

The complex was mixed with alumina (γ-Al₂O₃) in powder form, heated to 250° C., and maintained at this temperature for 3 hours to perform reduction and heat treatment. Then, it was dried and pulverized to prepare a catalyst for a gas sensor.

### Experimental Example 1

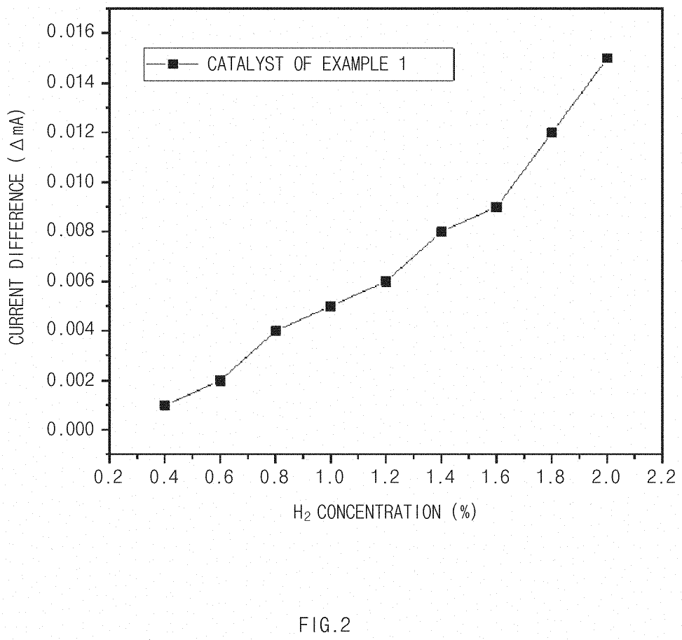

Using the catalyst for the gas sensor of Example 1, a current difference depending on concentration of hydrogen (H₂) was measured in a contact combustion method. The results are shown in FIG. 2.

As shown in FIG. 2, as the hydrogen concentration increases, the amount of hydrogen reacting with the catalyst increases. Thus, an accompanying exothermic reaction also increases, thereby increasing resistance of the electrode. From this, it was confirmed that the catalyst for the gas sensor of the present disclosure shows the current difference increasing in proportion to the hydrogen concentration, which has a linear fit (R-square=0.96).

To be used for a hydrogen gas sensor, it was seen that the catalyst for the gas sensor of the present disclosure is suitable for a hydrogen gas sensor, which should have a linear current difference depending on the hydrogen concentration.

### Comparative Example 1

A powdery alumina (γ-Al₂O₃) was mixed in a colloidal aqueous solution of Pt nanoparticles (concentration 4.5×10⁻⁵ mol) dispersed in 5 ml of deionized water and dried to prepare a support by containing Pt in alumina.

### Experimental Example 2

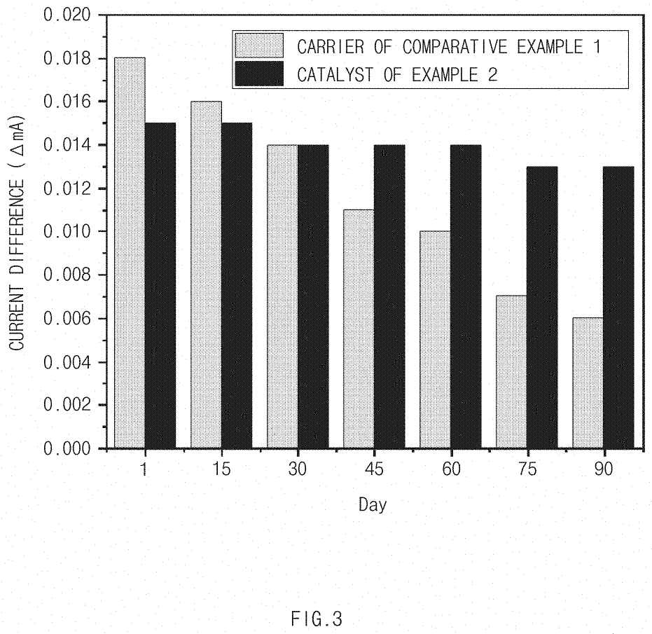

Using the catalyst for the gas sensor of Example 2 and the support of Comparative Example 1, the difference in current for 2% hydrogen concentration was measured for 90 days in a contact combustion method. The results are shown in FIG. 3.

As shown in FIG. 3, it was seen that the carrier of Comparative Example 1 tends to decrease the difference in the reaction current value for the hydrogen gas of the same concentration over time. This is a result of the exothermic reactivity due to the reaction with hydrogen gas decreasing over time. Thus, the activity of the catalyst carrier decreases due to the influence of the external environment.

On the other hand, the catalyst containing Pt-silica, which is the core-shell form of Example 2, partially reduced the reactivity with hydrogen gas, but showed a tendency to be maintained for a long period of time. From this, it was found that the catalyst for the gas sensor of the present disclosure has excellent long-term stability.

The catalyst for the gas sensor according to the present disclosure prevents deformation due to moisture existing in the external and surrounding environment. The catalyst also prevents movement and growth of the metal particles due to deterioration. Thus, the sensor lasts a long time and has excellent durability. Therefore, the catalyst for the gas sensor is very suitable for application to a hydrogen sensor that detects the concentration of the hydrogen gas in the hydrogen electric vehicle and checks for leaks, an alcohol sensor to measure whether or not you have been drinking, an indoor air quality sensor that measures the concentration of volatile organic compounds (VOC) in the vehicle, and the like.

Hereinabove, although the present disclosure has been described with reference to specific embodiments and the accompanying drawings, the present disclosure is not limited thereto. The embodiments and the disclosure may be variously modified and altered by those having ordinary skill in the art to which the present disclosure pertains without departing from the spirit and scope of the present disclosure claimed in the following claims.

What is claimed is:

1. A catalyst for a gas sensor comprising:
a support and a core-shell type complex contained in the support,
wherein the complex includes a metal-containing core and a porous nanostructured shell.

2. The catalyst for a gas sensor of claim 1, wherein the shell includes a plurality of pores having an average diameter of less than 10 nm.

3. The catalyst for a gas sensor of claim 1, wherein the shell includes at least one selected from a group consisting of silica, zeolite and starch.

4. The catalyst for a gas sensor of claim 1, wherein the metal includes at least one selected from a group consisting of platinum (Pt), palladium (Pd), gold (Au), nickel (Ni), ruthenium (Ru), copper (Cu), and rhodium (Rh).

5. The catalyst for a gas sensor of claim 1, wherein the complex includes a lipophilic functional group on a surface of the complex.

6. The catalyst for a gas sensor of claim 5, wherein the lipophilic functional group is derived from at least one selected from a group consisting of aluminic ester and aluminum hydroxide.

7. The catalyst for a gas sensor of claim 1, wherein the complex includes the core having an average diameter of 1 to 12 nm and the shell having an average thickness of 10 to 100 nm.

8. The catalyst for a gas sensor of claim 1, wherein the support is a ceramic including at least one selected from a group consisting of aluminum (Al), silicon (Si), boron (B), beryllium (Be), tin (Sn), zinc (Zn), tungsten (W), Cu, and magnesium (Mg).

9. The catalyst for the gas sensor of claim 1, wherein the Brunauer, Emmett, and Teller (BET) specific surface area is 50 to 300 m²/g.

10. The catalyst for a gas sensor of claim 1, wherein the complex includes a lipophilic functional group derived from at least one selected from a group consisting of aluminic ester and aluminum hydroxide on a surface of the complex.

11. A contact combustion type gas sensor comprising the catalyst for a gas sensor according to claim 1.

12. The contact combustion type gas sensor of claim 11, wherein the contact combustion type gas sensor detects at least one gas selected from a group consisting of hydrogen gas, ethanol gas, formaldehyde gas, and hydrocarbon (CₓHₓ) gas.
